(19) United States
(12) Patent Application Publication
(10) Pub. No.: US 2012/0330291 A1
(43) Pub. Date: Dec. 27, 2012

(54) NONLINEAR OPTICAL PHOTODYNAMIC THERAPY (NLO-PDT) OF THE CORNEA

(75) Inventors: James V. Jester, Irvine, CA (US); Tibor Juhasz, Corona del Mar, CA (US); Donald J. Brown, Santa Clarita, CA (US); Dongyul Chai, Costa Mesa, CA (US); Moritz Winkler, Irvine, CA (US)

(73) Assignee: THE REGENTS OF THE UNIVERSITY OF CALIFORNIA, Oakland, CA (US)

(21) Appl. No.: 13/523,058

(22) Filed: Jun. 14, 2012

Related U.S. Application Data
(60) Provisional application No. 61/500,801, filed on Jun. 24, 2011.

(51) Int. Cl. A61F 9/01

(52) U.S. Cl. 606/5

(57) ABSTRACT

The embodiments include method of nonlinear optical photodynamic therapy of tissue including the steps of providing pulsed infrared laser light for two-photon excited fluorescence tissue exposure, and selectively focusing the pulsed infrared laser light within the tissue at a focal plane to activate a photosensitizing agent to generate free radicals within a highly resolved axial and lateral spatial domain in the tissue. The invention is also directed to an apparatus for performing nonlinear optical photodynamic therapy of tissue including a pulsed infrared laser for providing two-photon excited fluorescence beam tissue exposure, a scanner for selectively and controllably moving the tissue and the beam relative to each other, and optics for selectively focusing the pulsed infrared laser light within the tissue at a point in a focal plane to activate a photosensitizing agent to generate free radicals within a highly resolved axial and lateral spatial domain in the tissue.

### RELATED APPLICATIONS

The present application is related to U.S. Provisional Patent Application Ser. No. 61/500,801, filed on Jun. 24, 2012, which is incorporated herein by reference and to which priority is claimed pursuant to 35 USC 119.

### GOVERNMENT RIGHTS

This invention was made with government support under EY0-7348 and EYO-18665 awarded by The National Eye Institute of the National Institute of Heath. The government has certain rights in the invention.

### BACKGROUND

1. Field of the Technology

The disclosure relates to the field of using nonlinear optical photodynamic therapy (NLO-PDT) to cause collagen crosslinking using infrared light and riboflavin in the cornea.

2. Description of the Prior Art

There are currently 87 papers that cite corneal collagen crosslinking. None of these papers cited the use of femtosecond lasers to activate photosensitizers in the cornea. There is one paper the evaluates collagen crosslinking following femtosecond laser generated tunnels in the cornea, but the researchers did not use the laser to activate the photosensitizer. In the past, crosslinking in the cornea has used UV light to activate the photosensitizer, riboflavin. The disadvantage of this approach is that it uses nonfocused light, which broadly and nonspecifically generates free radicals throughout the tissue volume, wherever the light penetrates.

### BRIEF SUMMARY

The illustrated embodiments of the invention are directed to apparatus and methods of using nonlinear optical (NLO), femtosecond-near infrared lasers used to activate photosensitizing chemicals in the cornea for various corneal treatments including corneal stiffening to treat corneal ectasia, refractive errors and astigmatism as well as provide antimicrobial and tumorcidal effects.

In particular the illustrated embodiments are directed to a method of nonlinear optical photodynamic therapy of tissue including the steps of providing pulsed infrared laser light for multiphoton tissue exposure, and selectively focusing the pulsed infrared laser light within the tissue at a focal plane to activate a photosensitizing agent to generate free radicals within a highly resolved axial and lateral spatial domain in the tissue.

The method may further include the step of pretreating the tissue with the photosensitive agent prior to selectively focusing the pulsed infrared laser light within the tissue. The photosensitive agent includes but not limited to riboflavin.

The step of providing pulsed infrared laser light includes providing near-infrared light to minimize cellular damage by reducing photon energy level of the laser light and increasing depth penetration into the tissue.

In the embodiment where the tissue is a cornea the step of selectively focusing the pulsed infrared laser light within the tissue includes providing sufficient intensity and length of irradiation to cause collagen crosslinking (CXL) effective for corneal stiffening.

In the embodiment where the tissue is a cornea the step of selectively focusing the pulsed infrared laser light within the tissue includes providing sufficient intensity and length of irradiation to effectively provide anti-microbial mediation to treat a corneal infection.

In the embodiment where the tissue is a cornea the step of selectively focusing the pulsed infrared laser light within the tissue includes providing sufficient intensity and length of irradiation to effectively inhibit corneal swelling in bullous keratopathy.

In the embodiment where the tissue is a cornea the step of selectively focusing the pulsed infrared laser light within the tissue includes providing sufficient intensity and length of irradiation to effectively kill cells, bacteria, tumors or neovascular vessels growing into the avascular cornea.

In one embodiment the step of selectively focusing the pulsed infrared laser light within the tissue includes providing sufficient intensity and length of irradiation to effectively activate the photosensitizing agent only at the focal plane.

In the embodiment where the tissue is a cornea the step of selectively focusing the pulsed infrared laser light within the tissue includes providing sufficient intensity and length of irradiation to effectively cause corneal stiffening by collagen crosslinking to precisely stiffen weakened corneas, including keratoconus and post-LASIK ectasia.

In the embodiment where the tissue is a cornea the step of selectively focusing the pulsed infrared laser light within the tissue includes providing sufficient intensity and length of irradiation to effectively cause corneal stiffening, flattening and steepening to precisely stiffen, flatten and steepen regions of the cornea to treat astigmatism and refractive errors associated with myopia, hyperopia and presbyopia.

In the embodiment where the tissue is a cornea the step of selectively focusing the pulsed infrared laser light within the tissue includes providing sufficient intensity and length of irradiation to effectively treat bacterial, fungal, and amoebic infections of the eye without antibiotics.

In the embodiment where the tissue is a cornea the step of selectively focusing the pulsed infrared laser light within the tissue includes providing sufficient intensity and length of irradiation to effectively kill labeled tumor cells in the eye following loading with photosensitizing dyes.

In the embodiment where the tissue is a cornea the step of selectively focusing the pulsed infrared laser light within the tissue includes providing sufficient intensity and length of irradiation to effectively treat clinical diseases including keratoconus, post-LASIK ectasia, astigmatism, myopia, hyperopia, infection and ocular tumors.

The embodiments of the invention also include an apparatus for performing nonlinear optical photodynamic therapy of tissue including a pulsed infrared laser for providing multiphoton tissue exposure, a scanner for selectively and controllably moving the tissue and the beam relative to each other, and optics for selectively focusing the pulsed infrared laser light within the tissue at a point in a focal plane to activate a photosensitizing agent to generate free radicals within a highly resolved axial and lateral spatial domain in the tissue.

The pulsed infrared laser light includes a near-infrared laser to minimize cellular damage by reducing energy level of the laser light and increasing depth penetration into the tissue.

In the embodiment where the tissue is a cornea the pulsed infrared laser is arranged and configured with the optics to provide sufficient intensity and length of irradiation to cause collagen crosslinking (CXL) effective for corneal stiffening, selective activation of anti-microbial medication to treat a corneal infection, inhibition of corneal swelling in bullous keratopathy, or selective killing of cells, bacteria, tumors or neovascular vessels growing into the avascular cornea.

In the embodiment where the tissue is a cornea the selectively focused pulsed infrared laser is arranged and configured with the optics to provide sufficient intensity and length of irradiation to effectively cause corneal stiffening by collagen crosslinking to precisely stiffen weakened corneas, including keratoconus and post-LASIK ectasia.

In the embodiment where the tissue is a cornea the selectively focused pulsed infrared laser is arranged and configured with the optics to provide sufficient intensity and length of irradiation to effectively cause corneal stiffening and flattening to precisely stiffen and flatten regions of the cornea to treat astigmatism and refractive errors associated with myopia, hyperopia and presbyopia.

In the embodiment where the tissue is a cornea the selectively focused pulsed infrared laser is arranged and configured with the optics to provide sufficient intensity and length of irradiation to effectively treat bacterial, fungal, and amoebic infections of the eye without antibiotics, or to effectively kill labeled tumor cells in the eye following loading with photosensitizing dyes.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a schematic diagram of an apparatus in which the invention may be practiced or embodied.

FIG. 2a is a side cross-sectional view of a microphotograph of an NLO treated rabbit cornea. FIG. 2b is a comparative graph of the autofluorescence of a UVA and an NLO treated rabbit cornea.

FIGS. 3a and 3b are a side cross-sectional view and a cutaway perspective view respectively of a jig where in the elasticity of gels subject to the method of the invention are measured.

FIGS. 4a-4c are diagrammatic depictions of the apparatus and the scanning pattern by which the gels are irradiated using nonlinear optical photodynamics with a pulsed infrared laser light for two-photon excited fluorescence.

FIG. 5 is a data scan of a gel using second harmonic generation to determine its thickness.

FIG. 6 is a graph of the indenting force verses the indenting depth for the gels before and after irradiation according to the methodology of the invention.

FIG. 7 is a graph of the elastic modulus of the gels comprised of a control group, a UVA exposed gel, a low power (100 mW) nonlinear optic (NLO) exposed gel and a high power (150 mW) nonlinear optic (NLO) exposed gel.

FIG. 8 is a graph of the increase in ratio of post to baseline elasticity of the treated gels comprised of a control group, a UVA exposed gel, a low power (100 mW) nonlinear optical (NLO) exposed gel and a high power (150 mW) nonlinear optical (NLO) exposed gel.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

It is known that collagen crosslinking can be caused using UV light and riboflavin in the cornea and that there is a correlation between collagen autofluorescence induced by crosslinking and the mechanical stiffening effects of UV-riboflavin. Autofluorescence is the natural emission of light by biological structures, such as mitochondria and lysosomes, when they have absorbed light, and is used to distinguish the light originating from artificially added fluorescent markers (fluorophores). We have established that collagen autofluorescence can be used to evaluate collagen crosslinking and that the intensity of autofluorescence is correlated with the amount of corneal stiffening. We have further developed preliminary data showing the NLO-PDT can induce increased corneal stromal autofluorescence in riboflavin soaked corneas. We also have data showing that NLO-PDT increases collagen gel stiffness, showing the proof of concept.

NLO-PDT uses very short pulsed laser light that can be accurately focused within tissues to activate photosensitizing chemicals such as riboflavin to generate free radicals within highly resolved spatial domains, axially and laterally. The very short-pulsed laser light used by NLO-PDT allows for precise focusing of high intensity light within very small volumes leading to nonlinear effects through multiple photon interactions. NLO-PDT allows for the use of lower energy laser light in the near-infrared region that has deeper depth of tissue penetration to activate photosensitizing chemicals that are normally activated by short wavelength, higher energy light that can cause cellular damage and has short depth penetration into tissues. Photosensitizers such as riboflavin that are excited by ultraviolet light (UV) are currently being used to treat corneal thinning by inducing oxygen radical generation leading to collagen crosslinking (CXL) and corneal stiffening. Additionally UV-CXL has been used as an anti-microbial method to treat corneal infections and to inhibit corneal swelling in bullous keratopathy.

A major drawback of UV-CXL is that there is no control over the volume of tissue where free radicals are generated when conventional UV light is used. This can lead to unwanted effects including cellular damage below the region of cross linking that may involve the corneal endothelium which is a nonregenerative cell layer in the cornea that is responsible for maintaining corneal transparency and limits the volume available for crosslinking. Therefore, the purpose of using NLO photoactivation is to generate free radicals only in the focal volume of the laser beam where NLO effects occur. This volume can be precisely controlled by lenses/objectives used to focus the light into the tissue, thereby leading to highly localized photoactivation.

NLO-PDT will allow for precise depth and area activation of photosensitizers that conventional UV-CXL lacks. Generation of free radicals by NLO femtosecond lasers can also be used to kill cells, bacteria, tumors and neovascular vessels growing into the avascular cornea with more precision then current approaches. The advantage of the disclosed NLO-PDT methodology is that activation of photosensitizer will occur only at the focal plane defined by the focusing objective of the laser. This will allow precise localization of oxygen radical generation and corneal crosslinking and anti-microbial and tumorcidal activity, as well as crosslinking in deeper corneal layers without damaging the corneal endothelium.

There are at least four immediate uses for localized NLO-PDT. First, collagen crosslinking and corneal stiffening can be used to more precisely stiffen weakened corneas, such as keratoconus and post-LASIK ectasia. Currently UV crosslinking is used clinically to treat these diseases. The disclosed approach will replace the current standard of care. Second, since crosslinking results in corneal stiffening and compensatory flattening and steepening in different regions, the disclosed NLO-PDT method can be used to precisely stiffen, flatten and steepen regions of the cornea to treat astigmatism and refractive errors associated with myopia, hyperopia and presbyopia. Third, the disclosed NLO-PDT methodology can be used to treat bacterial, fungal, and amoebic infections of the eye without antibiotics. Generation of free radicals is already used to sterilize tissue and fluids. NLO-PDT based oxygen radical generation can be used in a similar therapeutic modality with the disclosed methodology. Fourth, the disclosed NLO-PDT methodology can be used to kill labeled tumor cells in the eye following loading with photosensitizing dyes. The disclosed NLO-PDT methodology can be used to treat a range of clinical diseases including keratoconus, post-LASIK ectasia, astigmatism, myopia, hyperopia, infection and ocular tumors.

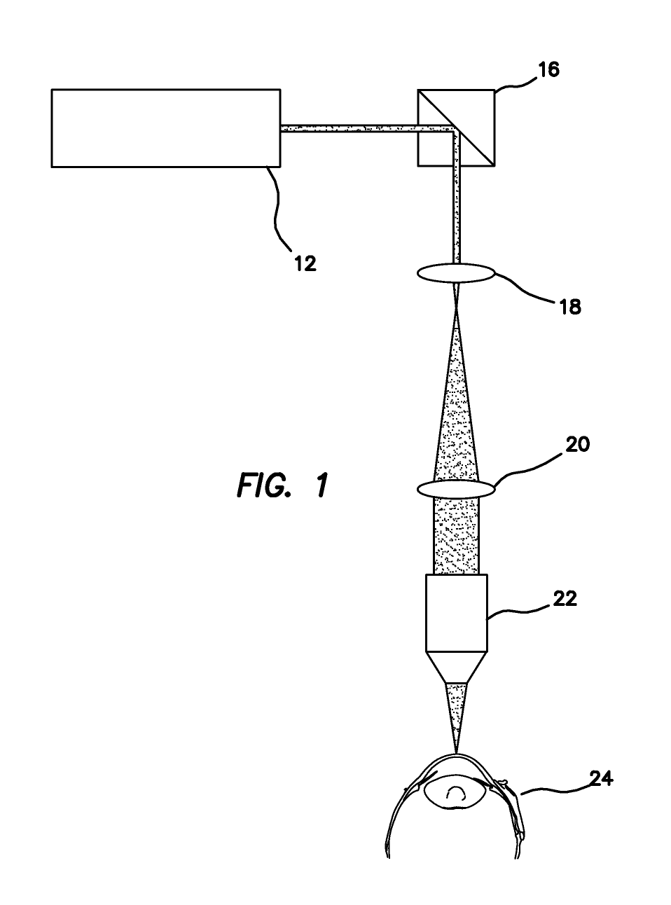

FIG. 1 is a simplified block diagram of an apparatus 10 implementing one embodiment of the invention. Femtosecond infrared pulsed laser 12 has a tunable output 14 from 700 to 960 nm that is scanned by an x/y scan unit 16 through a beam expander (lenses 18 and 20) and focusing optics 22 into a cornea 24. For experimental purposes the focusing optics is a conventional objective able to selectively focus the pulsed light into a volume of 22 μm³ located at 5.5 mm below the objective tip. Depth and volume of focus can be selectively manipulated by modification and movement of the focusing optics 22.

Two-photon excited fluorescence (TPEF) occurs when a fluorophore absorbs two or more photons of near-infrared light (700 to 960 nm) and emits a visible light photon. Two-photon excited fluorescence differs from single photon excited fluorescence (SPEF) in that the two-photon excited fluorescence signal is generated only at the focal plane, is less phototoxic than single-photon excited fluorescence, exhibits dramatically improved axial resolution and has a deeper depth of tissue penetration.

In an experiment illustrating the disclosed embodiment, fresh enucleated rabbit eyes were treated with 0.1% riboflavin in a 20% dextran solution by volume for 30 minutes. The eyes were moved relative to the objective 22 using an x-y translational stage with lateral movement of 10 mm/sec with a 3 μm line separation. The central cornea region was exposed to 760 nm Chameleon femtosecond laser light at 190 mW using laser 12 and a 20× objective 22. The axial position of the beam focus was controlled by moving the eye relative to the fixed focal plane defined by objective 22. The corneas were then removed, fixed and evaluated for TPEF collagen autofluorescence, which was measured using a Zeiss multiphoton confocal microscope.

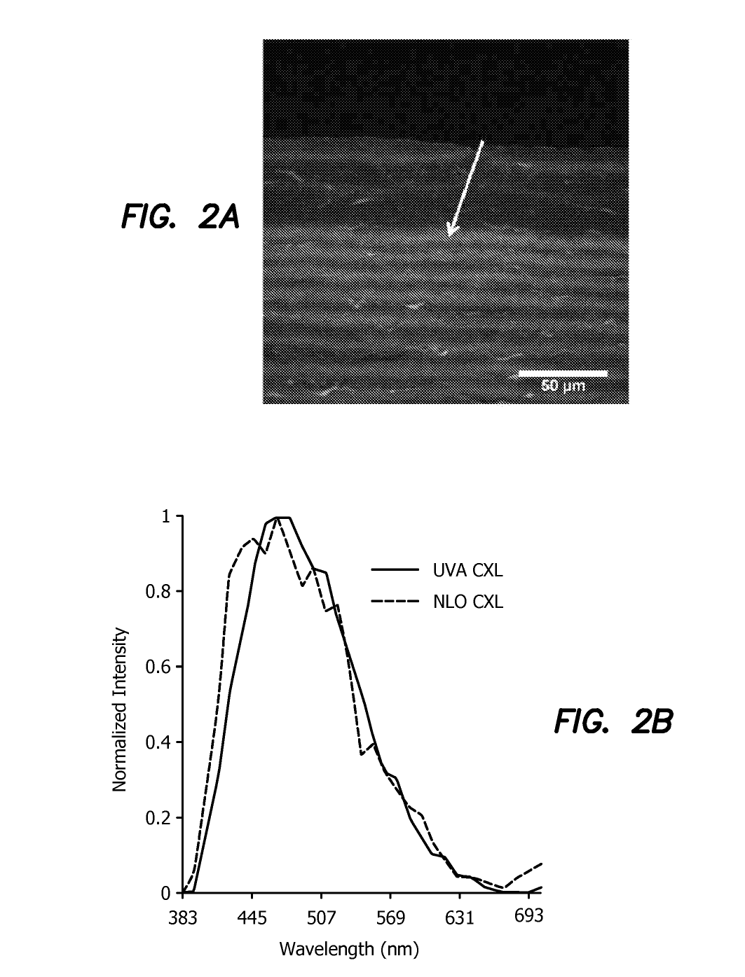

Multiphoton excitation of riboflavin within the corneal stroma generated fluorescence and free radicals leading to collagen crosslinking. NLO-PDCxI induced collagen autofluorescence within 9 1 mm line scans with 3 μm line separation is shown in the TPEF image shown in FIG. 2a. The NLO-PDCxI autofluorescence spectrum is shown graphically in FIG. 2b and compared against UVA collagen crosslinking autofluorescence in the cornea after 30 minutes irradiation in FIG. 2b. The normalized collagen autofluorescence spectrum generated by NLO-PDCxI as shown in FIG. 2b is very similar to the autofluorescence spectrum generated by UVA crosslinking. Therefore, selectively focused femtosecond laser beams can be used to create collagen crosslinking and corneal stiffening with similar biological effects as are observed with the more uncontrolled UVA induced crosslinking of the prior art.

In another demonstration of the concept of the invention collagen hydrogels were made and their mechanical stiffening using the methodology of the invention was measured. Compressed collagen hydrogels were made by polymerizing 3 ml of rat-tail type-1 collagen gel (3 mg/ml) in a 24 well tissue culture plate. Gels were compressed to 100 μm thickness using conventional methods. To facilitate transport, gels were compressed onto #54 Whatman Filter discs having a central 7.6 mm diameter hole exposing the hydrogel for biomechanical testing and NLO CXL.

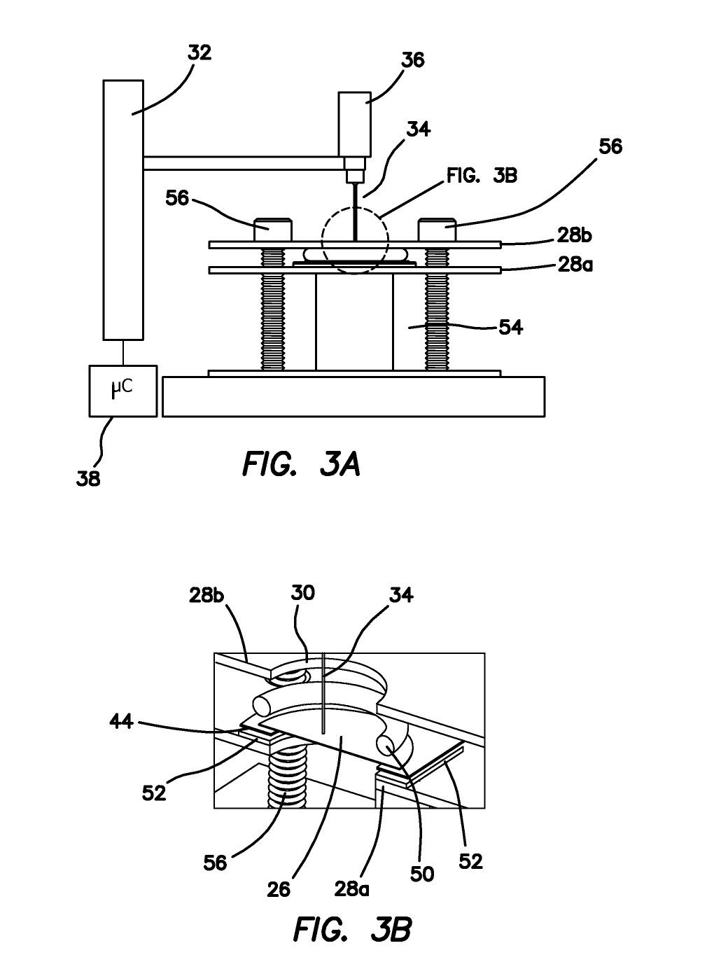

As shown in FIGS. 3a and 3b a jig was made to measure the elastic modulus of the gels 26, which were clamped between two steel plates 28a and 28b, each having a 7.6 mm diameter central hole 30 on a three dimensional control assembly 32. Plate 28a is mounted on a hollow transparent cylinder 54. Gel 26 is mounted on filter paper 44 and gasket 52 on top of plate 28a, each including a central hole 30 as best shown in FIG. 3b. An O-ring 50 is mounted on top of gel 26 followed by plate 38b. Plates 28a and 28b are bound together by compression from bolts 56. Gels 26 were then indented using a 250 μm diameter round tipped probe 34, as shown in FIG. 3b, attached to a force transducer 36 driven by automated electrical step motor within control assembly 32 controlled and recorded by computer 38 as shown in FIG. 3a. Each gel 26 was indented at the center through 1 mm depth at the velocity of 20 μm/sec and indenting force and depth recorded every 0.05 sec through 10 cycles. The elastic modulus, E, was then calculated using Equation 1, which is the modified Schwerin point-load solution of elastic modulus.

$$E = \frac{{( f ( υ ) )}^{3} {a}^{2} P}{{δ}^{3} h} , \\ f ( υ ) \approx 1.049 - 0.146 υ - 0.158 {υ}^{2} \qquad ( 1 )$$

Where P is the indenting force, a the radius of hole 30, h the gel thickness, v the Poisson ratio, and δ the indenting depth.

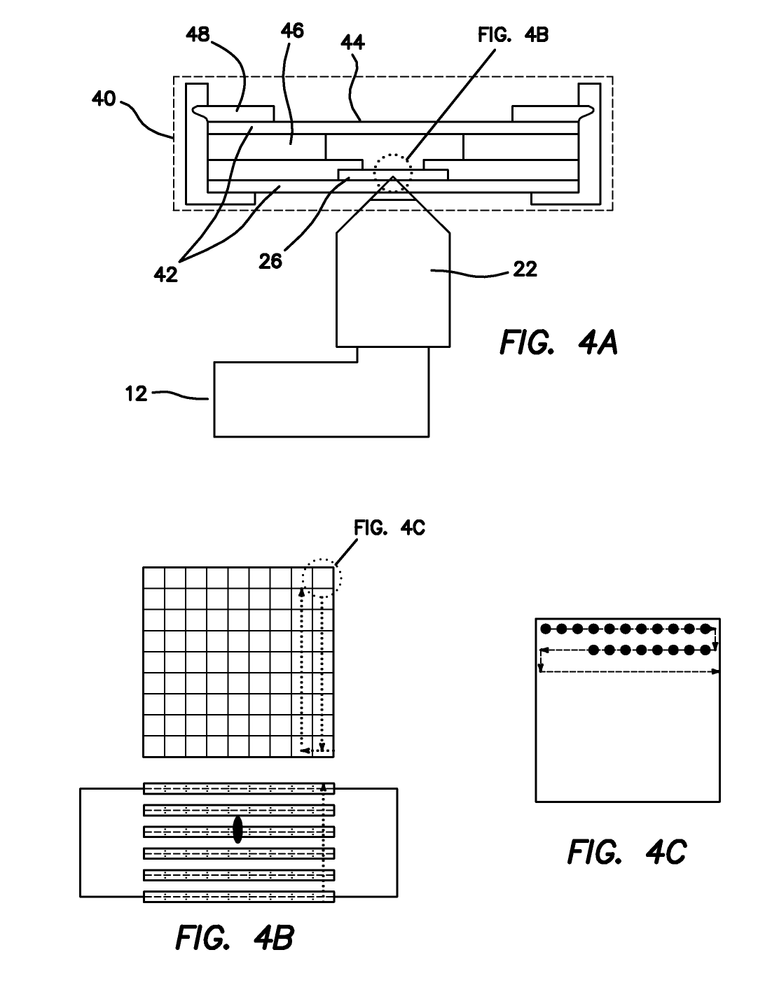

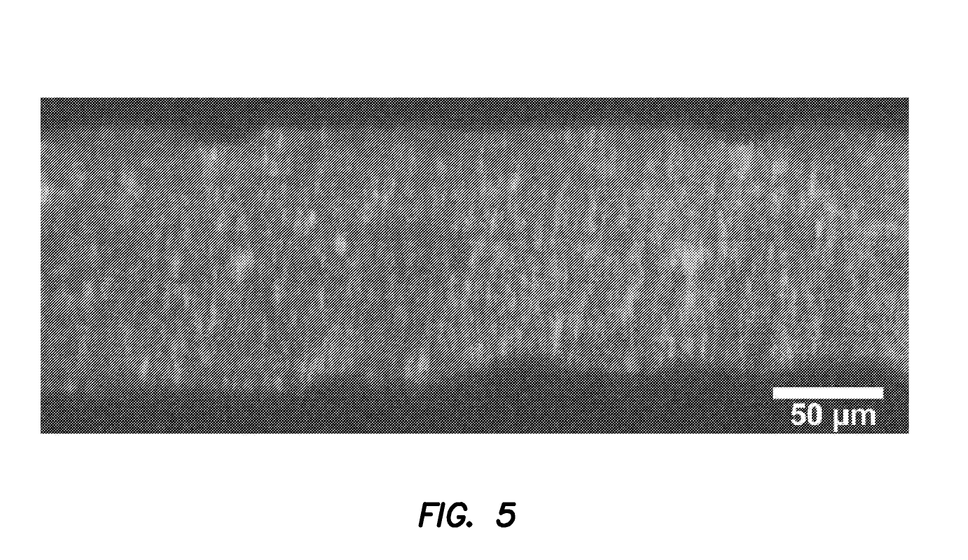

Gels 26 were then soaked in 0.1% riboflavin in phosphate buffered saline (PBS) and mounted in an NLO crosslinking chamber 40 as shown in FIG. 4a. The chambers 40 were then mounted onto a Zeiss 510 Meta confocal laser scanning microscope (CLSM) and gel thickness measured by second harmonic generation (SHG) imaging as shown in FIG. 5. NLO CXL was then performed by focusing a 100 mW (NLO I) or a 150 mW (NLO II), 760 nm femtosecond laser beam into the gel 26 using a 20× Zeiss apochromat objective lens 22 (NA=0.75). Gels 26 were scanned at 27.8 cm/sec velocity over a 5.2 mm×5.2 mm square area through the gel at 2 μm steps in a three dimensional tile scan as shown in FIGS. 4b and 4c. Control and UVA CXL gels 26 were left in the chamber 40 for the same duration as NLO CXL. For UVA CXL gels 26 were removed from the chamber 40 and exposed to 370 nm UVA light at 3 mW/cm² for 30 min over the same area as NLO CXL. The indenting force was then re-measured for each gel 26 as well as gel thickness.

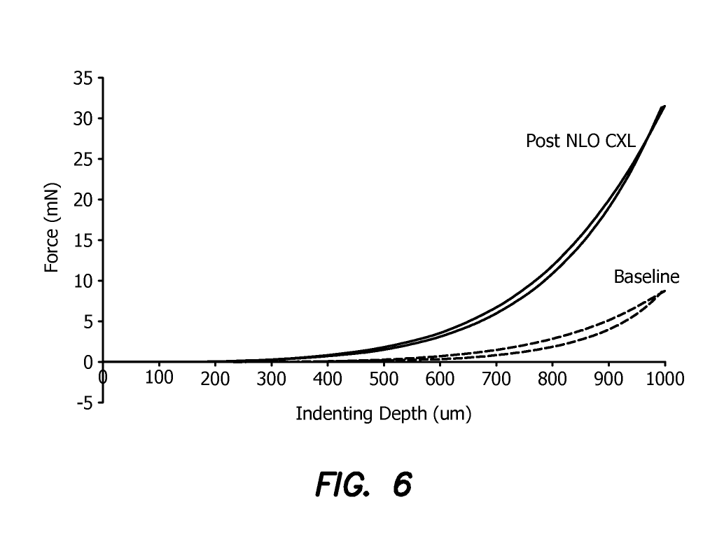

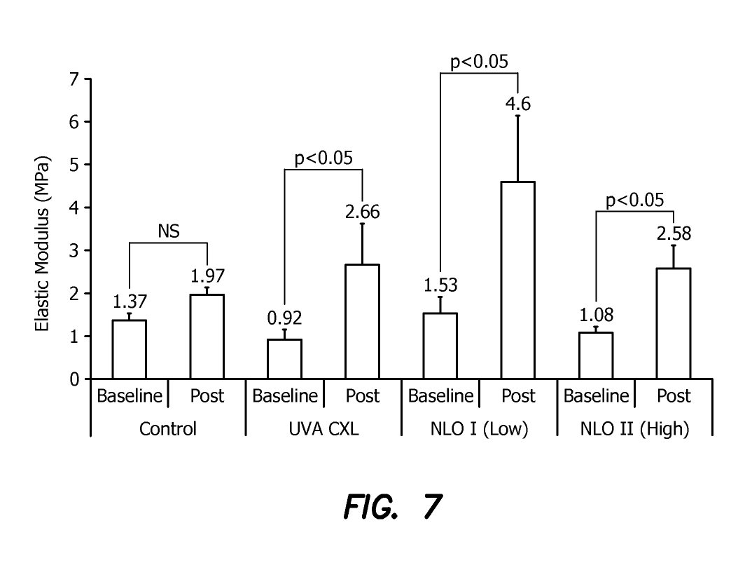

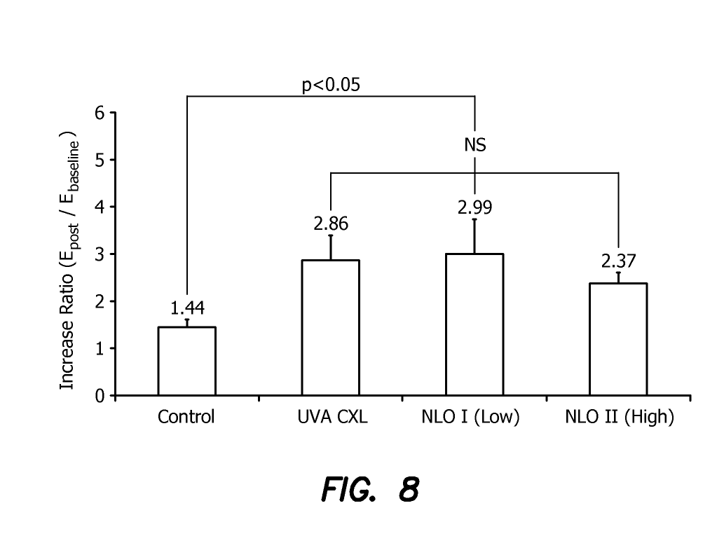

NLO collagen hydrogel crosslinking is shown in FIG. 6 at the 10^{th} cycle. NLO I treatment resulted in a marked increase in the indenting force suggesting that CXL and stiffening were induced by NLO I treatment. FIG. 7 shows baseline and post-treatment E values for each group before and after. Significantly increased post-treatment E values (p<0.05) were observed for all of CXL treatment groups. No significant difference was detected in the control group (p=0.22). Comparison of the ratio in E values between pre and post CXL (FIG. 8) showed no significant difference between UVA CXL and NLO CXL (p=0.38):

We thus show that nonlinear optical, multiphoton excitation of riboflavin using a femtosecond laser can induce collagen hydrogel crosslinking and mechanical stiffening similar to UVA CXL. Increased collagen autofluorescence in the cornea suggests that NLO CXL can stiffen the cornea. Because of the higher axial resolution of multiphoton processes, NLO CXL provides a safer and more effective therapeutic approach to treating corneal ectasia.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A method of nonlinear optical photodynamic therapy of tissue comprising:
providing pulsed infrared laser light for multiphoton tissue exposure; and
selectively focusing the pulsed infrared laser light within the tissue at a focal plane to activate a photosensitizing agent to generate free radicals within a highly resolved axial and lateral spatial domain in the tissue.

2. The method of claim 1 further comprising pretreating the tissue with the photosensitive agent prior to selectively focusing the pulsed infrared laser light within the tissue.

3. The method of claim 2 where the photosensitive agent comprises riboflavin.

4. The method of claim 1 where providing pulsed infrared laser light comprises providing near-infrared light to minimize cellular damage by reducing energy level of the laser light and increasing depth penetration into the tissue.

5. The method of claim 1 where the tissue is a cornea and where selectively focusing the pulsed infrared laser light within the tissue comprises providing sufficient intensity and length of irradiation to cause collagen crosslinking (CXL) effective for corneal stiffening.

6. The method of claim 1 where the tissue is a cornea and where selectively focusing the pulsed infrared laser light within the tissue comprises providing sufficient intensity and length of irradiation to effectively provide anti-microbial mediation to treat a corneal infection.

7. The method of claim 1 where the tissue is a cornea and where selectively focusing the pulsed infrared laser light within the tissue comprises providing sufficient intensity and length of irradiation to effectively inhibit corneal swelling in bullous keratopathy.

8. The method of claim 1 where the tissue is a cornea and where selectively focusing the pulsed infrared laser light within the tissue comprises providing sufficient intensity and length of irradiation to effectively kill cells, bacteria, tumors or neovascular vessels growing into the avascular cornea.

9. The method of claim 1 where selectively focusing the pulsed infrared laser light within the tissue comprises providing sufficient intensity and length of irradiation to effectively activate the photosensitizing agent only at the focal plane.

10. The method of claim 1 where the tissue is a cornea and where selectively focusing the pulsed infrared laser light within the tissue comprises providing sufficient intensity and length of irradiation to effectively cause corneal stiffening by collagen crosslinking to precisely stiffen weakened corneas, including keratoconus and post-LASIK ectasia.

11. The method of claim 1 where the tissue is a cornea and where selectively focusing the pulsed infrared laser light within the tissue comprises providing sufficient intensity and length of irradiation to effectively cause corneal stiffening, flattening and steepening to precisely stiffen, flatten and steepen regions of the cornea to treat astigmatism and refractive errors associated with myopia, hyperopia and presbyopia.

12. The method of claim 1 where the tissue is a cornea and where selectively focusing the pulsed infrared laser light within the tissue comprises providing sufficient intensity and length of irradiation to effectively treat bacterial, fungal, and amoebic infections of the eye without antibiotics.

13. The method of claim 1 where the tissue is a cornea and where selectively focusing the pulsed infrared laser light within the tissue comprises providing sufficient intensity and length of irradiation to effectively kill labeled tumor cells in the eye following loading with photosensitizing dyes.

14. The method of claim 1 where the tissue is a cornea and where selectively focusing the pulsed infrared laser light within the tissue comprises providing sufficient intensity and length of irradiation to effectively treat clinical diseases including keratoconus, post-LASIK ectasia, astigmatism, myopia, hyperopia, presbyopia, infection and ocular tumors.

15. An apparatus for performing nonlinear optical photodynamic therapy of tissue comprising:
a pulsed infrared laser for providing multiphoton tissue exposure;
a scanner for selectively and controllably moving the tissue and the beam relative to each other; and
optics for selectively focusing the pulsed infrared laser light within the tissue at a point in a focal plane to activate a photosensitizing agent to generate free radicals within a highly resolved axial and lateral spatial domain in the tissue.

16. The apparatus of claim 15 where the pulsed infrared laser light comprises a near-infrared laser to minimize cellular damage by reducing energy level of the laser light and increasing depth penetration into the tissue.

17. The apparatus of claim 15 where the tissue is a cornea and where the selectively focused the pulsed infrared laser is arranged and configured with the optics to provide sufficient intensity and length of irradiation to cause collagen crosslinking (CXL) effective for corneal stiffening, anti-microbial mediation to treat a corneal infection, inhibition of corneal swelling in bullous keratopathy, or selective killing of cells, bacteria, tumors or neovascular vessels growing into the avascular cornea.

18. The apparatus of claim 15 where the tissue is a cornea and where the selectively focused the pulsed infrared laser is arranged and configured with the optics to provide sufficient intensity and length of irradiation to effectively cause corneal stiffening by collagen crosslinking to precisely stiffen weakened corneas, including keratoconus and post-LASIK ectasia.

19. The apparatus of claim 15 where the tissue is a cornea and where the selectively focused the pulsed infrared laser is arranged and configured with the optics to provide sufficient intensity and length of irradiation to effectively cause corneal stiffening, flattening and steepening to precisely stiffen, flatten and steepen regions of the cornea to treat astigmatism and refractive errors associated with myopia, hyperopia and presbyopia.

20. The apparatus of claim 15 where the tissue is a cornea and where the selectively focused the pulsed infrared laser is arranged and configured with the optics to provide sufficient intensity and length of irradiation to effectively treat bacterial, fungal, and amoebic infections of the eye without antibiotics, or to effectively kill labeled of tumor cells in the eye following loading with photosensitizing dyes.
